(19) United States
(12) Patent Application Publication
(10) Pub. No.: US 2014/0378968 A1
(43) Pub. Date: Dec. 25, 2014

(54) MEDICAL DEVICES FOR RENAL NERVE ABLATION

(71) Applicant: BOSTON SCIENTIFIC SCIMED, INC., Maple Grove, MN (US)

(72) Inventors: Derek C. Sutermeister, Ham Lake, MN (US); Martin R. Willard, Burnsville, MN (US); Timothy A. Ostroot, Cokato, MN (US); Joel R. Munsinger, Blaine, MN (US); Kenneth R. Larson, Grand Rapids, MN (US); Prabodh Mathur, Laguna Niguel, CA (US)

(21) Appl. No.: 14/311,056

(22) Filed: Jun. 20, 2014

Related U.S. Application Data
(60) Provisional application No. 61/838,119, filed on Jun. 21, 2013.

(51) Int. Cl. A61B 18/14

(52) U.S. Cl. CPC A61B 18/1492; A61B 2018/1465; A61B 2018/00434
USPC 606/41

(57) ABSTRACT

Medical devices and methods for making and using the same are disclosed. An example medical device may include a medical device for ablation procedures. The medical device may include an elongate shaft having a distal region. An expandable member may be coupled to the distal region. One or more active electrode assemblies may be coupled to the expandable member. The one or more of the active electrode assemblies may include a resistive element adjacent the expandable member. The electrode assembly may include or may be used with microheaters.

### CROSS-REFERENCE TO RELATED APPLICATIONS

This application claims priority under 35 U.S.C. §119 to U.S. Provisional Application Ser. No. 61/838,119, filed Jun. 21, 2013, the entirety of which is incorporated herein by reference.

### TECHNICAL FIELD

The present disclosure pertains to medical devices, and methods for manufacturing medical devices. More particularly, the present disclosure pertains to medical devices for ablation procedures.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a medical device for renal nerve ablation. The medical device may include an elongate shaft having a distal region. An expandable member may be coupled to the distal region. A flexible electrode assembly may be coupled to the expandable member and in electrical connection with a control unit. In some cases, the flexible electrode assembly may include one or more electrodes that each include a resistive member. One or more active electrodes may be coupled to the expandable member.

Another example medical device may include a catheter shaft. An expandable balloon may be coupled to the catheter shaft. A plurality of flexible electrode assemblies may be coupled to the expandable balloon and a control unit may be coupled to the flexible electrode assemblies. The control unit coupled to the flexible electrode assemblies may be capable of energizing the flexible electrode assemblies with direct current.

Methods for ablating tissue adjacent a vessel lumen (e.g., ablating renal nerves) are disclosed. An example method may include providing a medical device. The medical device may include a catheter shaft, an expandable member coupled to the catheter shaft, and a plurality of flexible electrode assemblies coupled to the expandable member. The method may also include advancing the medical device through a blood vessel lumen, and activating at least one of the flexible electrode assemblies to heat a tissue at a target area with a resistive element of the activated one or more of the flexible electrode assemblies.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative medical device;

FIG. 2 is a schematic side view of a portion of an illustrative medical device;

FIG. 3 is a schematic side view of a portion of an illustrative medical device;

FIG. 4 is a schematic side view of a portion of an illustrative medical device;

FIGS. 5A and 5B are schematic side views of portions of illustrative medical devices;

FIG. 6 is a schematic side view of a portion of an illustrative medical device;

FIG. 7 is a schematic side view of a portion of an illustrative medical device; and

FIG. 8 is a schematic flow diagram showing an illustrative method of using a medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Certain treatments are aimed at the temporary or permanent interruption or modification of select nerve function. One example treatment is renal nerve ablation, which is sometimes used to treat conditions such as or related to hypertension, congestive heart failure, diabetes, or other conditions impacted by high blood pressure or salt retention. The kidneys produce a sympathetic response, which may increase the undesired retention of water and/or sodium. The result of the sympathetic response, for example, may be an increase in blood pressure. Ablating some of the nerves running to the kidneys (e.g., disposed adjacent to or otherwise along the renal arteries) may reduce or eliminate this sympathetic response, which may provide a corresponding reduction in the associated undesired symptoms (e.g., a reduction in blood pressure).

While the devices and methods described herein are discussed relative to renal nerve ablation and/or modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, pulmonary vein ablation, tumor ablation, benign prostatic hyperplasia therapy, nerve excitation or blocking or ablation, modulation of muscle activity, hyperthermia or other warming of tissues, etc.

Typical RF ablation therapies for treating hypertension and other medical issues may involve the use of highly conductive electrodes for high frequency RF ablation to generate heat. These highly conductive electrodes may be used to transfer electrical current to tissue of a patient. The tissue of a patient has an impedance to electrical energy, which leads to resistive heating of the tissue when the electrical energy comes into contact with the tissue. Essentially, a patient's tissue may act as a resistor of a circuit between an active electrode and a ground electrode.

One or more of several factors may affect the amount of heat transferred to a patient's tissue from typical RF ablation therapies. For example, the distance between an active electrode and a ground electrode, the types of tissue, the variability of the electrical pathways, and other factors, all may directly or indirectly impact tissue impedance. In some instances, unintentional heating of tissue may be result due to variances in tissue impedance between the active electrodes and ground electrodes. In typical mono-polar RF technology, for example, there may be a high risk of unintended tissue burns due to the imprecise nature of placing an active electrode within a patient's body and positioning a ground electrode exterior the patient's body.

With this disclosure, it is contemplated that electrical energy dissipation may rely on electrode impedance or on the impedance of the electrode-tissue interface rather than dissipation from the impedance of tissue between the active and ground electrodes. Illustratively, whereas typical RF ablation therapies may use medical devices with conductive electrodes, the disclosed concept may include resistive flexible electrode assemblies including one or more of resistive electrodes, a resistive material coated on conductive electrodes, microheaters, and/or other resistors. The use of resistive flexible electrode assemblies, may allow for low voltage ablation devices, where the electrical energy may be provided via direct current or alternating current (e.g., low voltage direct current, low frequency (less than 200 KHz) alternating current, or other energy).

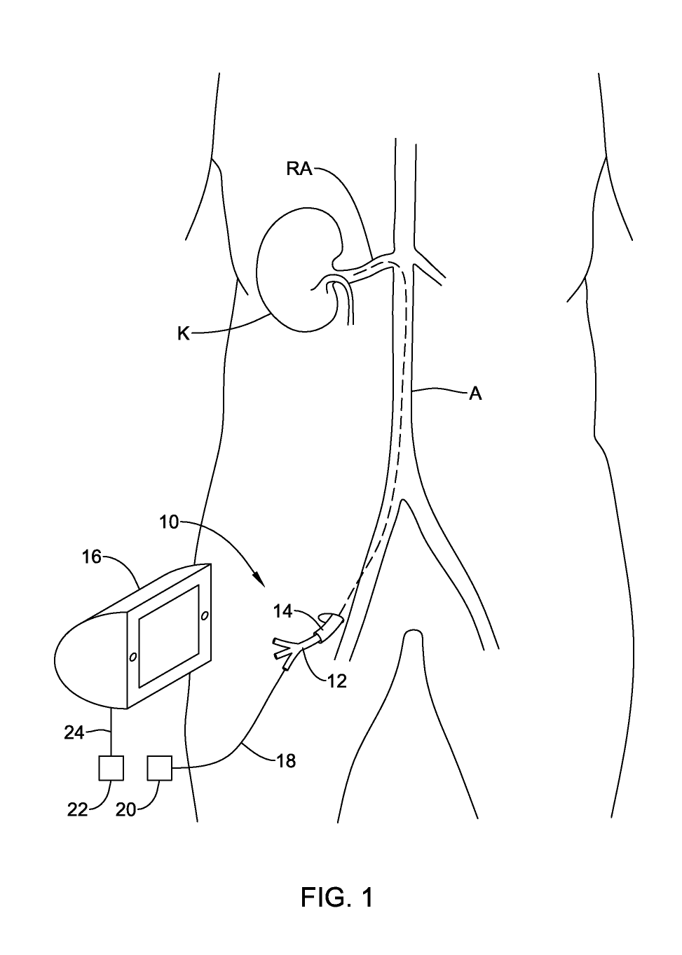

FIG. 1 is a schematic view of an example renal nerve modulation system 10. System 10 may include a medical device 12 (e.g., a renal nerve ablation medical device). The medical device 12 may be used to ablate nerves (e.g., renal nerves) disposed adjacent to the kidney K (e.g., renal nerves disposed about a renal artery RA). In use, the medical device 12 may be advanced through a blood vessel such as the aorta A to a position within the renal artery RA. This may include advancing the medical device 12 through a guide sheath or catheter 14. When positioned as desired, the medical device 12 may be activated to activate one or more electrodes (not shown). This may include coupling medical device 12 to a control unit 16 having or connected to a power source (e.g., a generator (not shown), a battery, etc.) so as to supply the desired activation energy to the electrodes and/or control the operation of the medical device 12. In some instances, the medical device 12 may include a wire or conductive member 18 with a connector 20 that may be connected to a connector 22 on control unit 16 and/or a wire 24 coupled to control unit 16. In at least some embodiments, control unit 16 may also be utilized to supply/receive the appropriate electrical energy and/or signal to activate one or more electrodes disposed at or near a distal end of the medical device 12. When suitably activated, the electrodes may be capable of ablating tissue (e.g., renal nerves or other tissue) as described below and one or more sensors located adjacent the electrodes may be used to sense physical and/or biological parameters, as desired.

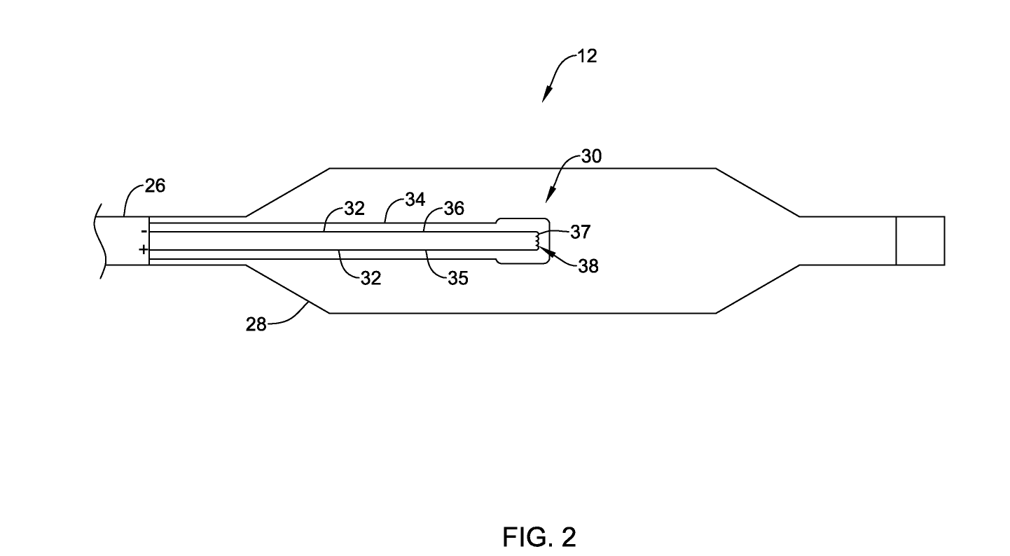

FIGS. 2-7 are side views illustrating a portion of the medical device 12. Here it can be seen that device 12 may include a tubular member 26 (e.g., a catheter shaft). An expandable member 28 may be coupled to the tubular member 26 (e.g., the expandable member 28 may be coupled to a distal end of the tubular member 26). In at least some embodiments, the expandable member 28 may be an expandable balloon (e.g., a compliant, non-compliant, or semi-compliant balloon) that may be capable of being folded. In instances where the expandable member 28 is an expandable balloon, the expandable balloon may be made out of polyether block amide (e.g., PEBAX™), polyethylene terephthalate (PET), or other similar or dissimilar materials. In other embodiments, expandable member 28 may be or may include, among other features, a basket, a stent, a plurality of struts, or the like.

In some instances, the expandable member 28 may be formed of a single layer of material or multiple layers of material. Illustratively, a multitude of thin layers (e.g., two layers, three layers, four layers, five layers, six layers, seven layer, eight layers, nine layers, or more layers) may distribute stresses and defects, such as cracks or punctures, so that they are less likely to propagate through the wall to the point of causing failure of the medical device 12 than they might be in an expandable member 28 including a wall with a single layer of material. In some instances, the layers of the balloon may be thicker (e.g., individually or in combination) than the typical size of defects (e.g., the size of gas bubbles formed in the extrusion of the layers or the size of foreign particles in the material). In some examples, an expandable member 28 having multiple layers may take the form of one or more of the embodiments discussed in U.S. Pat. No. 7,947,059, entitled MULTILAYER MEDICAL DEVICE, issued on May 24, 2011 to Chin et al., which is hereby incorporated by reference in its entirety for all purposes.

A flexible electrode assembly 30 (e.g., a flex circuit) comprising resistive members 38 may be applied to the expandable member 28 in any suitable manner. In some instances, the flexible electrode assemblies 30 may be printed onto the expandable member 28 through any known printing technique in the circuit or electrode assembly art. Some example flexible electrode assemblies 30 that may be utilized for device 12 (and/or other devices disclosed herein) may include or otherwise be similar to flex circuits disclosed in U.S. patent application Ser. No. 13/760,846, the entire disclosure of which is herein incorporated by reference for all purposes. For example, the flex circuit may include one or more polymeric layers (e.g., polyimide) with electrode(s) and conductive member(s) coupled thereto. In other embodiments, electrodes or resistive members 38 may be disposed along a printed circuit on the expandable member 28 or may be disposed on the expandable member 28 and connected to one or more wires (e.g., a solid or substantially solid resistive member 38 may be connected inside a strut of a basket to one or more conductive wires).

In at least some embodiments, the flexible electrode assembly 30 may be an ablation flexible electrode assembly that is capable of delivering thermal ablation energy to a suitable target. For example, the flexible electrode assembly 30 may be capable of delivering thermal ablation energy to tissue positioned adjacent to a blood vessel such as renal nerves positioned adjacent to a renal artery. Such flexible electrode assembly 30 may be electrically configured to be monopolar or bipolar, and/or have heat sensing capability.

In some instances, the flexible electrode assembly 30 may be able to localize thermal energy without completing an electrical pathway to an exterior of a patient's body. For example, the resistive coating 40 may be an electrically resistive material, but a thermally conductive material, such that when RF energy, direct current energy, alternating current energy, or other energy is passed through the resistive member 38 thermal energy (e.g., heat) is generated and passed onto a target area of a patient's body.

Illustratively, one or more flexible electrode assemblies 30 (e.g., flex circuits) may be coupled to the expandable member 28, as shown in FIGS. 2-6. The flexible electrode assembly 30 may include one or more resistive members 38 (e.g., resistors 37, resistive electrodes 41 or microheaters 42) and one or more conductive members 32 in electrical communication with a resistive member 38. The resistive members 38 (e.g., the resistors 37, the resistive electrodes 41 and/or the microheaters 42) may be configured to be mounted on the expandable member 28 or any other device (e.g., a probe, etc.). Illustratively, the resistive members 38 may be placed on the expandable member in more than one location, more than two locations, more than three locations, more than four locations, more than five locations, more than ten locations, more than fifteen locations, more than twenty locations, more than thirty locations, more than forty locations, more than fifty locations and/or no more than one-hundred locations. Alternatively, other number of locations for resistive members 38 may be utilized. In some instances, one or more resistive members 38 may be placed directly on or in the expandable member 28 through being printed, glued, and/or connected in any other manner to the expandable member 28 without a substrate. Alternatively, or in addition, one or more resistive member 38 may be formed on or connected to a substrate, such that the substrate may be adhered or otherwise affixed to or positioned within the expandable member 28.

In one example, as shown in FIG. 2, the flexible electrode assembly 30 may be applied to the expandable member 28 and two conductive members 32 may extend along an insulator layer 34 to a resistor 37. Illustratively, a first conductive member 32 may be an active trace 35 supplying power to the resistor 37 (e.g., a carbon resistor or other resistor) and a second conductive member 32 may be a return or ground trace 36. Such a flexible electrode assembly 30 may utilize bipolar technology to provide power to the resistive member 38 and heat a patient's target area.

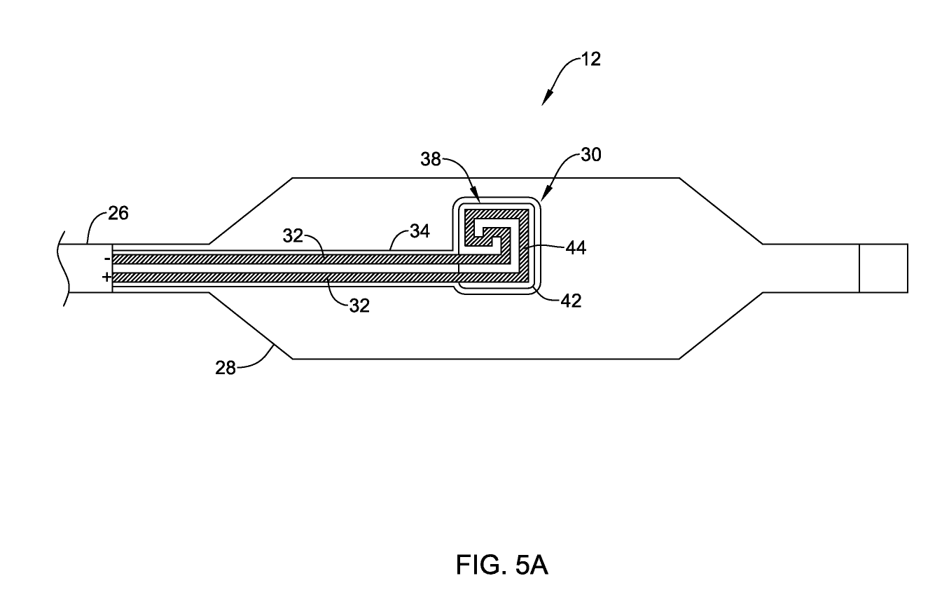

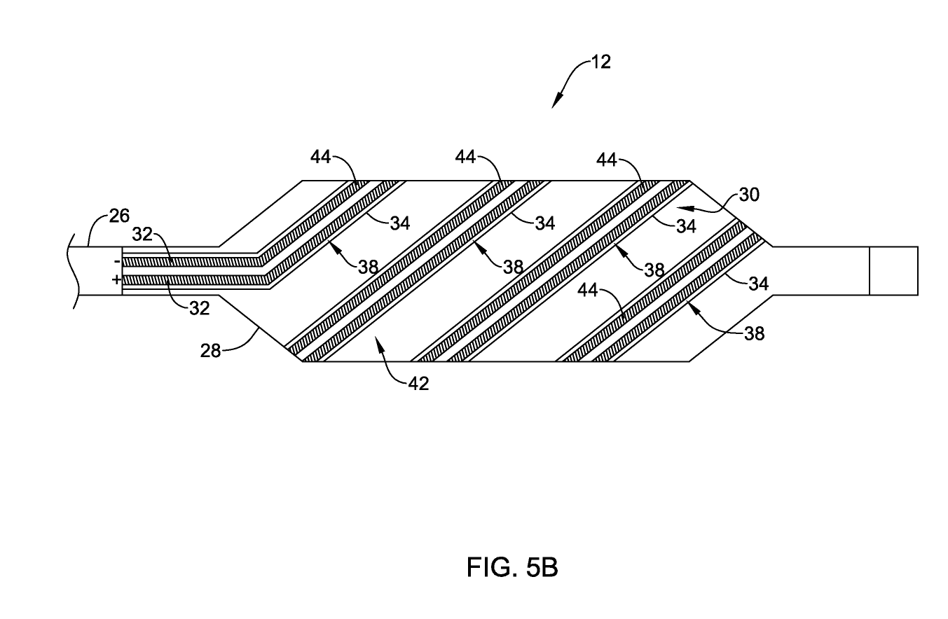

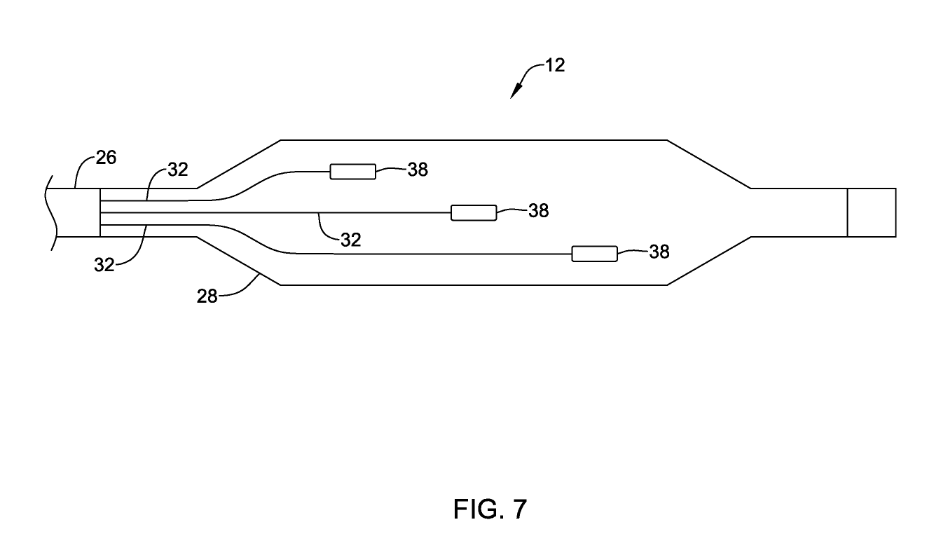

The resistive members 38 may be placed on the expandable member 28 to create one or more patterns. The patterns may be formed and/or designed to enhance efficacy and/or aid in the folding and/or the flexibility of the expandable member 28. Illustratively, the patterns of the resistive members 38 on the expandable member 28 may include, but are not limited to spiral designs (FIG. 5B), abstract heater designs (FIG. 7 with conductive members 32 and resistive members 38 applied directly to the expandable member 38), heating zone designs, balloon fold-friendly designs and/or other designs, as desired.

The resistive members 38 may allow for direct and/or radiant heating of a patient's tissue. Direct heating may include the resistive members 38 in direct contact with a patient's tissue (e.g., a vessel wall, etc.). Radiant heating may include heating with an exposed resistive member 38 that is not in direct contact with a patient's tissue or heating with a resistive member 38 that has a covering (e.g., a thin layer covering) thereon, such that the resistive member 38 indirectly heats a patient's tissue. In instances, where the expandable member 28 includes walls with multiple layers, one or more resistive members 38 may be placed between the multiple layers of the wall of the multi-layered expandable member 28 to provide radiant heat to a patient's tissue.

Alternatively, or in addition, the resistive members 38 may be applied on any device or feature having an electrical connection, as desired. In some instances, the resistive members 38 may be placed on the expandable member 28 directly, as discussed above or otherwise, on an expandable member 28 via a flexible circuit 30, as discussed above or otherwise, a separate polymer substrate, at the end of a probe, etc.

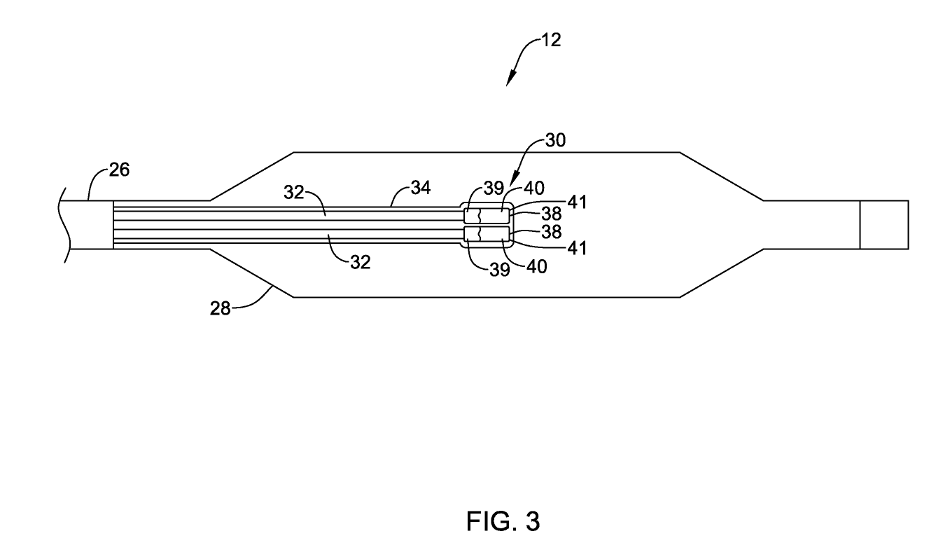

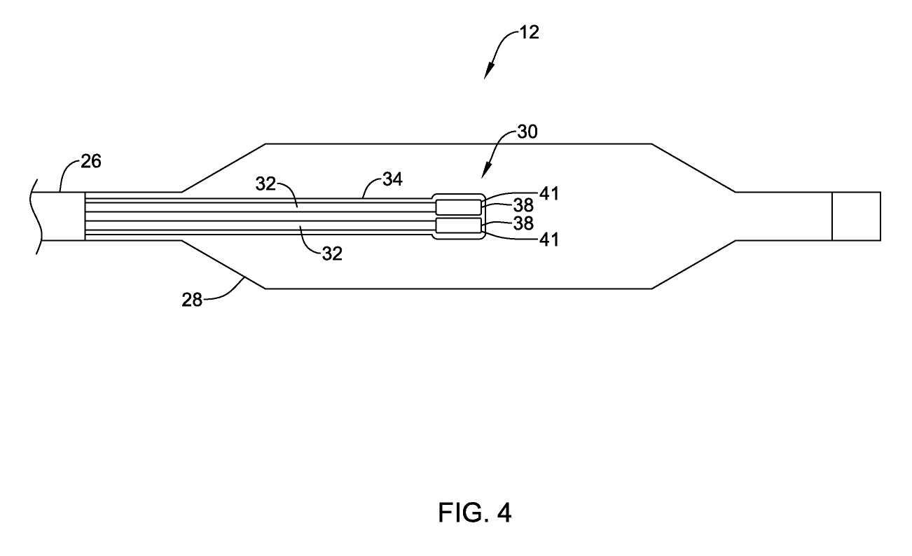

In some instances, the flexible electrode assemblies 30 may include a resistive member 38 that may be or may include a resistive electrode 41, as shown in FIGS. 3 and 4. Resistive electrodes 41 may be resistive due to their material properties or due to the material properties of coatings thereon (e.g., a resistive coating 40 on a conductive element 39). Any material may be used to create resistive electrodes 41, where the material may be highly electrically resistive, thermally conductive, and biocompatible. In one example, Nichrome material (e.g., Nichrome wire) may be utilized as a resistive electrode. Typically the resistivity of a material is determined by an electrical resistance of a uniform specimen of the material, as measured in ohms, multiplied by a cross-sectional area of the piece of material divided by the length of the piece of material. Illustratively, some materials that may be used to create resistive electrodes 41 may include, but are not limited to, materials with a positive thermal coefficient (PTC).

Resistive electrodes 41 may be utilized with current mono-polar and/or bi-polar ablation technologies and/or techniques. As such, the resistive electrodes 41 may utilize ablation technologies providing RF energy to an electrode, where the electrode has been modified to be a resistive electrode. Alternatively, or in addition, direct current, alternating current, or other energy may be provided to the resistive electrode, as desired.

In some instances, the resistive electrodes 41 may include a resistive coating 40 on a conductive element 39 thereof, where the resistive coating 40 may be capable of producing thermal ablation energy (e.g., heat) from electrical current passing through the conductive element 39, as shown in FIG. 3, to apply to tissue of a patient. In instances where the resistive electrode 41 may include a resistive coating 40, the conductive element 39 may be entirely, substantially entirely, or partially encapsulated with the resistive member 40.

Alternatively, the resistive electrode 41 may be formed from an electrically resistive and thermally conductive material, as shown in FIG. 4. For example, electrically resistive and thermally conductive materials may include, but are not limited to platinum, nickel, copper, or other material.

Alternatively, or in addition, to the flexible electrode assemblies 30 including a resistive member 38 that is a resistive electrode 41, the flexible electrode assemblies 30 may include one or more resistive members 38 that are microheater(s) 42 having one or more microheater paths 44, as shown in FIGS. 5A and 5B. The microheaters 42 may have any size and shape and may be able to generate various amounts of heat with low voltage levels. In one example, the microheaters 42 may be made as small as 0.001 inches by 0.001 inches or smaller and as large as twelve (12) inches by twelve (12) inches or larger. In one example, the microheaters 42 may be 0.008 inches by 0.008 inches. Although it is contemplated that some ablation procedures may only need medical devices 12 capable of generating heat up to a maximum temperature of 100 degrees Celsius, in a similar or different example, the microheaters 42 may be able to generate heat up to 200 degrees Celsius with just 2.5 volts of electricity in twenty (20) seconds. Although the microheaters 42 are described herein as having dimensions forming a square, the microheaters 42 may take on any shape and/or size, as desired.

In some instances, where the microheater is 0.008 inches by 0.008 inches, the microheater paths 44 may have a 0.001 inch width. In some instances, but not necessarily all, such a sized microheater 42 may be able to generate heat up to two hundred (200) degrees Celsius with 2.5 volts of electricity in twenty (20) seconds and/or generate heat up to ninety (90) degrees Celsius with 1.5 volts of electricity in five (5) seconds. These are just examples. Other dimensions and/or capabilities are contemplated.

The microheaters 42 may include microheater paths 44 formed in any manner, as desired. The microheaters 42 and microheater paths 44 may be formed from any, same or different, material, as desired. In one example, the microheaters 42 may be formed of (e.g., entirely of or at least partially formed of) a metal infused polymer composite, such as polydimethulsioxane (PDMS) with metal (e.g., silver) microsized particles poured into a mold with a specific microheater path 44 pattern.

Use of the microheater 42 may allow for the use of an energy source other than RF energy. In some instances, a low voltage direct current, low frequency (less than 200 KHz) alternating current, or other energy may be utilized as an alternative to the RF energy (with a frequency of 200 KHz or higher) used by typical renal nerve ablation medical devices. Such use of low voltage direct current may reduce the need for shielding conductive members 32 and/or the resistive members 38, may allow for use of conductive members 32 that form or have smaller diameter wires than wires required with non-resistive electrodes and/or RF energy, may allow for use of minimal, if any, electrical insulation around the conductive members 32, may allow for reduced size flexible electrode assemblies 30 when compared to the size of a flexible electrode assembly required for non-resistive electrodes and/or RF energy utilizing flexible electrode assembly, may reduced the likelihood of the electrical circuits shorting out and/or the electricity jumping and heating unintended areas of tissue, and/or may provide other advantages.

In some instances, the resistive member 38 may be or may include a resistive temperature detector (RTD). RTDs may be a sensor used to measure temperature by correlating the resistance of the RTD element with temperature. RTDs may have a linear resistance-to-temperature curve, which may be advantageous when controlling the temperature of a resistive member 38 during an ablation therapy. RTDs may be made from any material. For example, an RTD may be made from platinum, nickel, copper, or other material.

In some instances, the control unit 16 may provide the power to the conductive members 32 and the resistive members 38. The control unit 16 may receive power to be supplied to the resistive members 38 in one or more of several ways. In one example, for a microheater 42, the control unit 16 may need to supply as little as, or less than, approximately 1.6 volts up to, or more than, approximately 50 volts of direct current electricity. Illustratively, the control unit 16 may receive power from a battery and/or an alternating current (AC) power source (e.g., where the control unit 16, or other feature of the medical device 12, may include an AC/DC converter). In some instances, the AC power source may come directly from the wall or through a console, which may be converted to DC power at the control unit 16 or at any other location. The power from the battery source may come from a battery in the control unit 16, in a handle of the medical device 12, and/or a battery located elsewhere.

In instances where the medical device 12 is self-contained (e.g., the power source is within a handle of the medical device or otherwise self-contained), the medical device may be controlled by a tablet computing device or other computing device. Alternatively, the control unit 16 may be a tablet computing device or other computing device. The tablet computing device or other computing device, like the control unit 16, may include a memory and processor configured to effect an ablation therapy or other treatment with the medical device 12. The tablet computing device or other computing device may be connected to the medical device 12 (e.g., a handle of the medical device 12 or other portion thereof) in a wireless manner (e.g., through a BLUETOOTH™, WiFi connection, ZIGBEE™ or other wireless connection) or a wired manner (e.g., a USB connection or other wired connection).

Although a medical device 12 utilizing low voltage DC power with a microheater 42 may be operated with a control unit 16, the utilization of low voltage DC power may open the door to using various technologies for providing power to the microheater 42 or resistive electrodes 41 during ablation therapies or other treatments. In one instance, the medical device 12 may be self contained such that the power source (e.g., a battery) may be built into a handle of the medical device 12.

In some instances, the control unit 16 or other power source may be capable of passing between 0.1 volts and 50 volts of direct current to the flexible circuit assembly 30 along the conductive members 32. Illustratively, the control unit 16 may be capable of passing between 0.1 volts and 25 volts, 0.1 volts and 10 volts, 0.1 volts and 5 volts, 1 volt and 5 volts, 1 volt and 10 volts, 1 volt and 25 volts, 1 volt and 50 volts or any other range of voltages from the control unit 16 and the resistive members 38.

One or more conductive members 32 may be coupled to the resistive members 38 of the flexible electrode assembly 30. The conductive members 32 may take the form of a conductive trace, a conductive wire, or the like. Conductive member 32 may be coupled to or be a region of conductive member 18 and, ultimately, may be coupled to control unit 16. Thus, a suitable energy (e.g., RF energy, low voltage direct current, or other energy) may be delivered to the flexible electrode assembly 30 via the conductive member 32.

A thermally and/or electrically non-conductive or insulator layer 34 may be disposed adjacent to the conductive member 32 or a portion thereof. The resistive member 38 may be disposed along the thermally and/or electrically non-conductive layer 34 (e.g., at a distal end of the conductive member 32). Thermally and/or electrically non-conductive layer 34 may insulate the resistive member 38 and/or conductive member 32 from other structures including conductive features along the expandable member 28.

The conductive members 32 may be any shape and/or any size capable of carrying RF energy, direct current, alternating current, or other energy from the control unit 16 or other power source to the resistive members 38. For example, when using the resistive members 38, the medical device 12 may require low voltage levels to attain a required amount of heat for an ablation therapy and as a result, the conductive member 32 for passing energy from the control unit 16 to the resistive member 38 may have a small diameter such as a diameter less than 0.005 inches, less than 0.01 inches, less than 0.02 inches, less than 0.05 inches, less than 0.1 inches and so on. In one instance, when the conductive member 32 has a diameter less than about 0.005 inches, the conductive member 32 may be capable of passing direct current from the control unit 16 or other power source to the resistive members 38 to provide thermal energy to the a patient's target area.

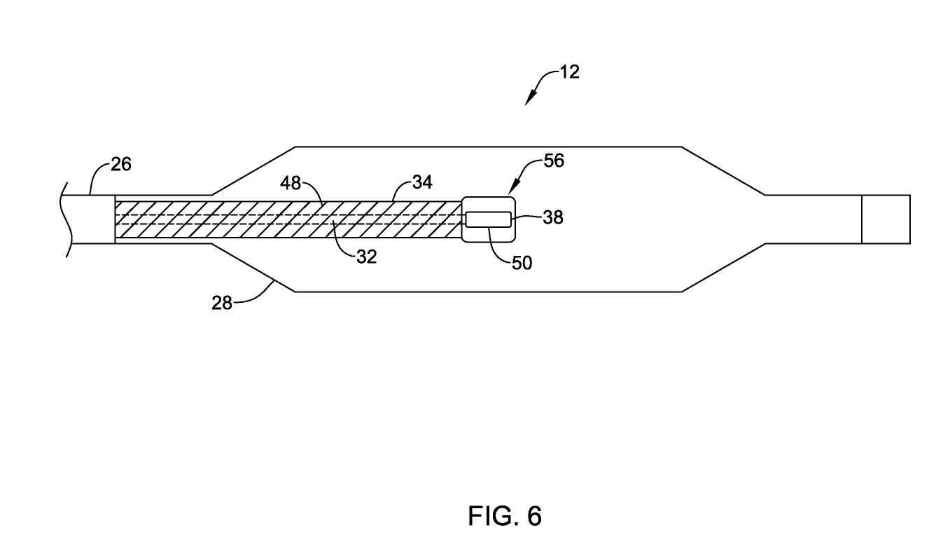

In some instances, one or more conductive members 32 or one or more portions of the conductive members 32 may have a PTC. A PTC may refer to materials that experience an increase in electrical resistance when their temperature is raised. Put another way, the higher the PTC, the greater an increase in electrical resistance for a given temperature increase. Materials that may have a PTC include, but are not limited to, materials made of doped polycrystalline ceramic which may contain barium titanate (BaTiO₃), materials made of plastic with carbon gains embedded therein, materials employing thermally sensitive silicon, and/or other materials having a PTC. The conductive members 32 having a PTC may form their own resistive member 38. For example, the conductive member 32 having a PTC may have a thermal insulation layer 48 covering all but a distal area 50 of the conductive member 32, where the exposed distal area 50 of the conductive member 32 may form the resistive member 38, as shown in FIG. 6. In the example, the exposed distal area 50 (e.g., resistive member 38) of the conductive member 32 with a PTC may heat a target area of a patient's tissue.

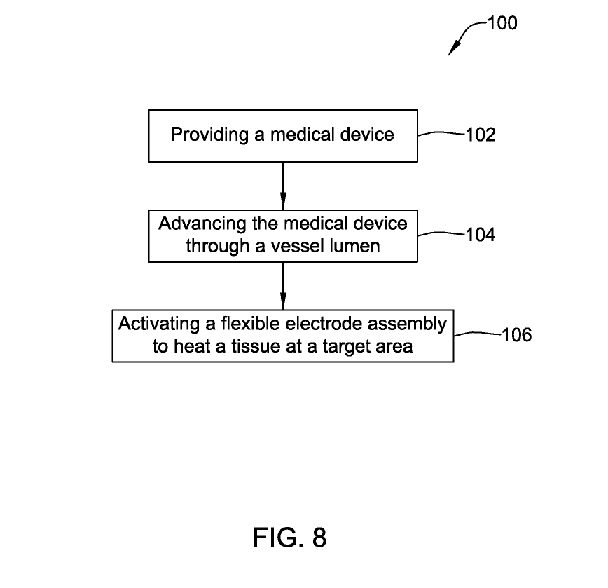

In operation, the renal nerve ablation medical device 12 may be used in a method 100 of ablating tissue adjacent a vessel wall, as shown in the schematic flow diagram of FIG. 8. Illustratively, a medical device 12 may be provided 102 that includes the guide sheath or catheter 14, an expandable member 28 that may be coupled to a tubular member or a catheter shaft 26, and a plurality of flexible electrode assemblies 30 or other electrode assemblies that may be coupled to the expandable member 28. The provided medical device 12 may be advanced 104 through a blood vessel to a position adjacent to a target tissue (e.g., within a renal artery). In some embodiments, the target tissue may be one or more renal nerves disposed about the renal artery RA. When suitably positioned, expandable member 28 may be expanded. This may place the flexible electrode assembly 30 adjacent or against the wall of the blood vessel. One or more resistive members 38 of the flexible electrode assembly 30 may be activated 106. Thermal energy may be transmitted from resistive members 38, through the target tissue (where renal nerves may be ablated, modulated, or otherwise impacted). In some instances, direct current may be applied to one or more of the flexible electrode assemblies 30 including the resistive member(s) 38 at or near a distal end of a tubular member or catheter shaft 26 to activate one or more of the electrodes and heat the tissue at the patient's target area.

Illustratively, during some procedures with the medical device 12, the power to the resistive members 38 may be controlled through the control unit 16 or other mechanism. In one example, the power to the resistive members 38 may be modulated via the control unit 16 to target a specified or determined temperature profile at the resistive members 38, as desired. In some instances, it may be desirable to utilize a slow rise temperature rise at the resistive members 38 or target tissue to prevent charring and/or popping at the contact surface between a target tissue and the medical device 12.

The materials that can be used for the various components of device 12 (and/or other devices disclosed herein) may include those commonly associated with medical devices. For simplicity purposes, the following discussion makes reference to device 12. However, this is not intended to limit the devices and methods described herein, as the discussion may be applied to other similar tubular members and/or components of tubular members or devices disclosed herein.

Device 12 and the various components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions of device of 12 may be doped with, made of, or otherwise include a radiopaque material. Radiopaque materials are understood to be materials capable of producing a relatively bright image on a fluoroscopy screen or another imaging technique during a medical procedure. This relatively bright image aids the user of device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 12 to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for performing ablation procedures, comprising:
a catheter shaft;
an expandable member coupled to the catheter shaft;
a flexible electrode assembly coupled to the expandable member, where the flexible electrode assembly includes one or more electrodes that each include a resistive member; and
a control unit coupled to the flexible electrode assembly.

2. The medical device of claim 1, wherein the flexible electrode assembly includes a monopolar electrode assembly.

3. The medical device of claim 1, wherein the flexible electrode assembly includes a bipolar electrode assembly.

4. The medical device of claim 1, wherein one or more resistive member forms a microheater.

5. The medical device of claim 1, wherein the flexible electrode assembly is capable of localizing thermal energy around the resistive member without having to complete an electrical pathway to an exterior of a patient's body.

6. The medical device of claim 1, wherein direct current is passed through the resistive member to provide thermal energy to a target area.

7. The medical device of claim 6, wherein the control unit is configured to deliver between 0.1 volts to 50 volts of direct current to the flexible electrode assembly.

8. The medical device of claim 1, wherein the flexible electrode assembly includes one or more wires with a diameter less than 0.005 inches.

9. The medical device of claim 1, wherein the flexible electrode assembly includes one or more wires with a positive thermal coefficient.

10. The medical device of claim 1, wherein the control unit is configured to energize the resistive member with approximately 2.5 volts or less.

11. The medical device of claim 1, further comprising:
a temperature sensor disposed adjacent to at least one of the one or more electrodes.

12. A medical device, comprising:
a catheter shaft;
an expandable balloon coupled to the catheter shaft;
a plurality of flexible electrode assemblies coupled to the expandable balloon;
a control unit coupled to the flexible electrode assemblies; and
wherein the control unit is configured to energize the flexible electrode assemblies with direct current.

13. The medical device of claim 12, wherein the flexible electrode assemblies are printed on the expandable balloon.

14. The medical device of claim 12, wherein the flexible electrode assemblies include one or more resistive electrodes.

15. The medical device of claim 12, wherein the plurality of flexible electrode assemblies includes a plurality of microheater paths.

16. The medical device of claim 15, wherein the microheater paths are patterned on the expandable balloon to aid in folding of the expandable balloon.

17. A method of ablating tissue adjacent to a vessel lumen, comprising:
providing a medical device, comprising:
a catheter shaft;
an expandable member coupled to the catheter shaft; and
a plurality of flexible electrode assemblies coupled to the expandable member, where the flexible electrode assemblies include one or more electrodes that each include a resistive member;
advancing the medical device through a vessel lumen; and
activating one or more of the flexible electrode assemblies to heat a tissue at a target area with the resistive member of the activated one or more of the flexible electrode assemblies.

18. The method of claim 17, further comprising:
applying direct current to the flexible electrode assemblies including the resistive member at a distal end of the catheter shaft to activate one or more of the one or more electrodes and heat the tissue at the target area.

19. The method of claim 17, wherein activating one or more of the flexible electrode assemblies to heat tissue at a target area with the resistive member of the activated one or more of the flexible electrode assemblies ablates tissue adjacent a pulmonary vein.

20. The method of claim 17, wherein activating one or more of the flexible electrode assemblies to heat tissue at a target area with the resistive member of the activated one or more of the flexible electrode assemblies ablates nerve tissue adjacent a renal artery.
